(19) United States
(12) Patent Application Publication
(10) Pub. No.: US 2010/0036470 A1
(43) Pub. Date: Feb. 11, 2010

(54) Laser-Based Fabrication of Implantable Stimulation Electrodes

(75) Inventor: Stefan B. Nielsen, Innsbruck (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/534,234

(22) Filed: Aug. 3, 2009

Related U.S. Application Data
(60) Provisional application No. 61/087,275, filed on Aug. 8, 2008.

(51) Int. Cl. A61N 1/05; B22F 7/04

(52) U.S. Cl. 607/137; 419/9

(57) ABSTRACT

A method of fabricating an implantable stimulation electrode is described. An arrangement of conductive metal powder is provided atop an electrode substrate. A laser beam is then focused on the metal powder to form the stimulation electrode in a given geometry with given dimensions and supported by the electrode substrate.

This application claims priority from U.S. Provisional Patent Application 61/087,275, filed Aug. 8, 2008, which is incorporated herein by reference.

### FIELD OF THE INVENTION

The present invention relates to medical implants, and more specifically to fabrication of stimulation electrodes for cochlear implant systems.

### BACKGROUND ART

Implantable neurostimulation electrodes are currently produced using manual and/or semi-automated methods for placing metal wires, metal traces, or stimulation contacts (all typically made of platinum or a platinum alloy) in electrically isolating material. This may be done manually, but manual work is very operator dependent and it is difficult to specify in sufficient detail to ensure reproducible results. Hand-made devices may therefore unintentionally be subject to significant variations in performance, which represents a technical problem. Furthermore, manual work is associated with extensive and time-consuming training of personnel.

Semi-automated processes can overcome some of these problems. Among the approaches currently used are photolithography, electroplating, and vapor depositioning of metal. Some conductive portions of the resulting structure may further be covered by thermal melt encapsulation or spin coating in an electrically insulating material as needed. These are described further, for example, in PCT Patent Application WO 2004064687, U.S. Patent Publication 2008027525, and U.S. Patent Publication 2006017273, which are incorporated herein by reference. Although precise and reproducible, these methods involve multiple individual procedural steps that include extensive use of chemicals, which may pollute materials that eventually are to be implanted, thus making purity control of chemicals a very important factor.

With existing electrode fabrication processes, it is possible to make structures in two dimensions (though quite difficult to control) and the height or thickness of the deposited metal is basically the same at different locations. But it is not practical to make three dimensional structures because physical masking of parts of the depositioning area—and therefore interruption of the process—would be needed. Thus, there are several technical problems involved with these methods.

Other semi-automated processes include removal of material from a sheet of metal to create predefined traces and pads. For example, U.S. Pat. No. 7,240,416 (incorporated herein by reference) suggests using embossing and electrical discharge machining or laser ablation. Embossing and selective material removal can facilitate making some three-dimensional structuring, but it may be limited by the thickness of the metal sheet used. Furthermore, it is generally not desirable to initially place a relatively large amount of metal (which is typically an expensive noble metal such as platinum or an alloy thereof) and then remove most of the metal to create individual traces and pads.

### SUMMARY OF THE INVENTION

A method of fabricating an implantable stimulation electrode is described. An arrangement of conductive metal powder is provided atop an electrode substrate. A laser beam is then focused on the metal powder to form the stimulation electrode in a given geometry with given dimensions and supported by the electrode substrate.

In some specific embodiments, the metal powder may be blown onto the electrode substrate so that the stimulation electrode is formed by laser deposition of the metal powder. In other embodiments, the metal powder may be pre-layered on the electrode substrate so that the stimulation electrode is formed by laser sintering of the metal powder.

The given geometry may specifically be a two-dimensional geometry so that the stimulation electrode lies substantially in a plane, or a three-dimensional geometry so that the stimulation electrode has a three-dimensional geometry. The stimulation electrode may be formed in a recess in the surface of the electrode substrate, or above the surface of the electrode substrate.

The stimulation electrode may include at least one electrode contact for delivering an electrical signal to adjacent tissue. The electrode contact may include a layer of conductive surface structures for increasing the surface area of the electrode contact. There may be at least one conductive trace element for delivering the electrical signal to the electrode contact. In some embodiments, there may be multiple conductive trace elements and/or multiple electrode contacts. The electrode contact and the conductive trace element may have the same height or different heights.

The method may further include smoothing at least one surface of the stimulation electrode to reduce insertion trauma, for example, by electro-polishing or chemical etching. The stimulation electrode may be formed into multiple different metal layers. The metal powder may include at least one of platinum, iridium, gold, silver, palladium, tungsten, or an alloy thereof. The electrode substrate may be at least one of silicon, glass, polymer, or metal.

In any of the foregoing, the stimulation electrode may specifically be a cochlear implant electrode. And embodiments of the present invention also include an implantable stimulation electrode formed by any of the foregoing methods.

### BRIEF DESCRIPTION OF THE DRAWINGS

FIG. 1A-B shows a process for laser deposition of an implantable stimulation electrode according to one embodiment of the present invention.

FIG. 2A-C shows a process for laser sintering of an implantable stimulation electrode according to one embodiment of the present invention.

FIG. 3A-C shows various two-dimensional stimulation electrodes according to embodiments of the present invention.

FIG. 4A-B shows embodiments of the present invention in which tissue contacts are higher than the conductive traces that connect to them.

FIG. 5A-B shows various three-dimensional stimulation electrodes according to embodiments of the present invention.

FIG. 6 shows an embodiment of the present invention in which the stimulation electrodes are embedded into recessed grooves in the electrode substrate.

### DETAILED DESCRIPTION OF SPECIFIC EMBODIMENTS

Laser depositioning of metal powder is a known technique for repairing metal parts (e.g. turbine blades) and for rapid prototyping. In this process, a laser beam is focused on a substrate (e.g. metal) and metal powder is blown onto the focus point. The laser energy heats the metal powder to create metal deposits on the underlying substrate. The process is controlled by parameters which include laser energy, laser spot size, and powder flow rate. When the laser and the powder injector are moved over the substrate, metal traces of a given thickness, width and height can be created. If the laser and the powder injector are moved up away from the substrate, three-dimensional structures can be created such as a spring shape standing up away from the substrate. This technology has been described for production of porous surfaces such as for bone implants (see, e.g., U.S. Patent Publication 2007202351, Canadian Patent 2,242,790, U.S. Patent Publication 2006073356, and U.S. Patent Publication 2002082741, which are incorporated herein by reference) and for depositing anti-microbial substances on implant surfaces (see, e.g., Patent Cooperation Treaty Publication WO 2008002750, which is incorporated herein by reference).

Selective laser sintering is similar to laser depositioning as discussed above, but instead of blowing in a metal powder, one or more layers of metal powder are pre-positioned on or in the substrate and sintered by the laser to create metal structures having a desired geometry and dimensions, depending in part on how many layers of metal powder are sintered.

Embodiments of the present invention use similar techniques to fabricate implantable stimulation electrodes. With existing laser technology, electrode structures can (theoretically) be created in widths and heights of down to ˜10 and ˜5 micrometers respectively. This level of detail is adequate for making high-density electrode arrays and conductors for implantation.

Laser depositioning of metal powder for making implantable stimulation electrodes can be flexible, simple, fast, reproducible, and highly automated. Relatively little equipment is needed and there are few process steps, most of which can be automated. Laser sintering on the other hand may be somewhat less flexible, but useful for making two-dimensional structures of limited height such as thin conducting trace elements such as wires and thin tissue contacts such as electrode contacts. Moreover, use of laser sintering avoids blowing possibly expensive noble metal powder around in the laser device. Depending on whether or not the excess powder can be re-collected and re-used, laser sintering may be more cost effective than laser depositioning.

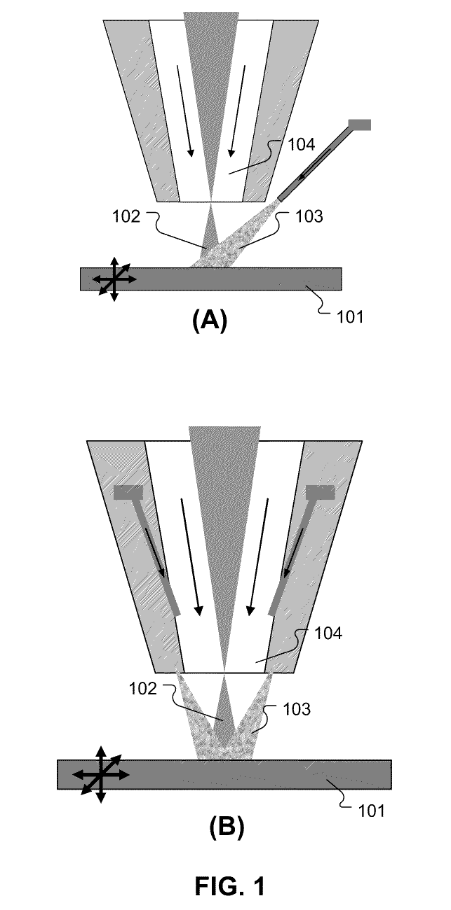

FIG. 1A-B shows one embodiment of the present invention based on a laser deposition process that creates an implantable stimulation electrode. An electrode substrate 101 is provided for mechanically supporting the stimulation electrode. A laser beam 102 is focused on a conductive metal powder 103 to form the stimulation electrode on the electrode substrate 101 in a given geometry with given dimensions. In the arrangement shown in FIG. 1A-B, a carrier gas 104 is fed around the perimeter of the laser beam 102 to control spattering by the metal powder 103 as it is fused by the laser beam 102. FIG. 1A shows a side feed arrangement where the metal powder 103 is fed by a carrier gas 104 to a desired location on the electrode substrate 101 where the laser beam 102 is focused. FIG. 1B shows an alternative arrangement where the metal powder 103 is fed by the carrier gas 104 in a coaxial arrangement around the laser beam 102.

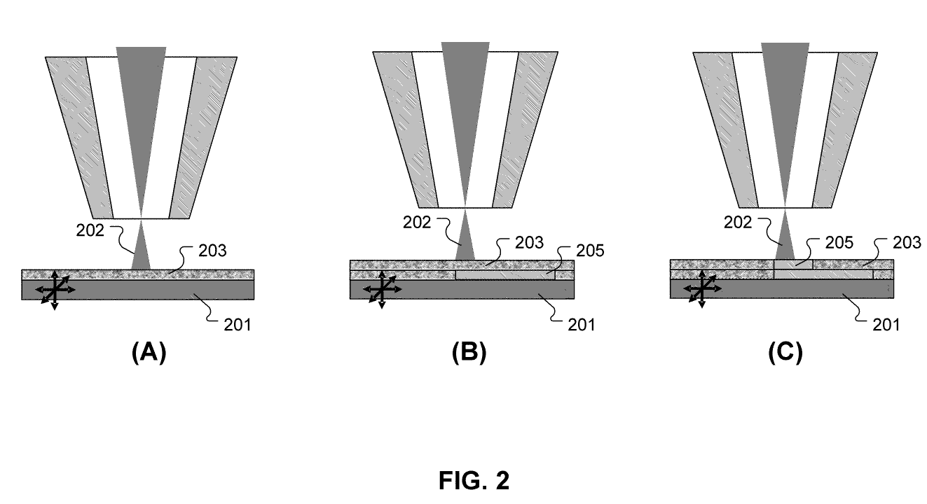

FIG. 2A-C shows a process for laser sintering of an implantable stimulation electrode according to one embodiment of the present invention wherein the metal powder 203 is pre-layered in one or more layers on the electrode substrate 201. In FIG. 2A, a single layer of metal powder 203 lies over the electrode substrate 201 and where the focus of the laser beam 202 travels, the powder 203 will fuse into a solid metal structure the size and shape of the beam travel pattern. A stimulation electrode 205 can be built up away from the electrode substrate 201 by repeating the process with successive further layers as shown in FIGS. 2B and 2C.

Using either laser deposition or laser sintering a stimulation electrode may be formed into multiple different metal layers. For example, a layer of gold can be deposited first, and then followed by a layer of platinum on top. Alternatively, the core of a stimulation electrode could be made of a less expensive material having desired material characteristics, e.g., stainless steel, and then an outer layer of platinum or platinum alloy may be applied by laser deposition or laser sintering. In this way the electrical characteristics (such as the charge injection properties) and the mechanical characteristics (such as strength and flexibly) can be tailored to meet desired design objectives. The metal powder may specifically be one or more of platinum, iridium, gold, silver, palladium, tungsten, or an alloy thereof. The electrode substrate may be at least one of silicon, glass, polymer, or metal. The method may further include smoothing at least one surface of the stimulation electrode to reduce insertion trauma, for example, by electro-polishing or chemical etching.

The given geometry may specifically be a two-dimensional geometry so that the stimulation electrode lies substantially in a plane, or a three-dimensional geometry so that the stimulation electrode has a three-dimensional geometry. The stimulation electrode may be formed in a recess in the surface of the electrode substrate, or above the surface of the electrode substrate. And the stimulation electrode may include at least one electrode contact for delivering an electrical signal to adjacent tissue. The electrode contact may further include a layer of conductive surface structures for increasing the surface area of the electrode contact. There may be at least one conductive trace element for delivering the electrical signal to the electrode contact. In some embodiments, there may be multiple conductive trace elements and/or multiple electrode contacts. The electrode contact and the conductive trace element may have the same height or different heights.

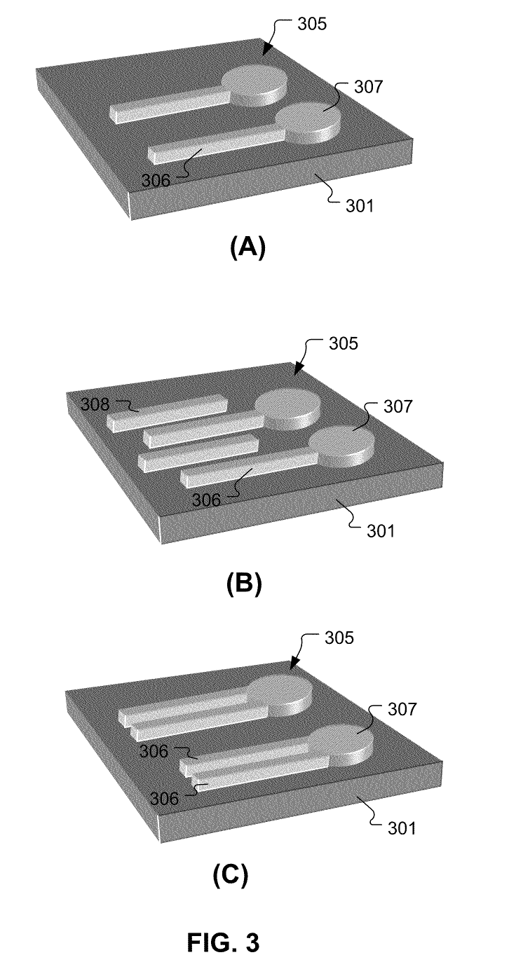

FIG. 3A-C shows various two-dimensional stimulation electrodes 305 according to embodiments of the present invention. In FIG. 3A, smooth conductive trace elements 306 (equivalent to electric wires) and electrode contacts 307 of the same height are created. FIG. 3B shows the addition of electrically passive mechanical supportive structures 308 which allow control of some of the mechanical properties of the finished stimulation electrode 305. In FIG. 3C, more than one conductive trace element 306 connects to each electrode contact 307.

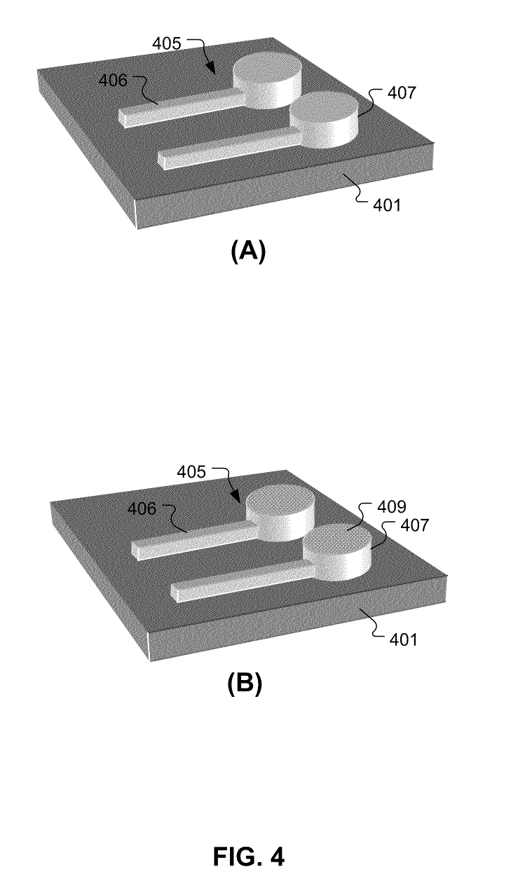

FIG. 4A-B shows embodiments of the present invention in which the electrode contacts 407 are higher than the conductive traces 406 that connect to them. FIG. 4A shows smooth conductive traces 406 and electrode contacts 407 where the electrode contacts 407 are higher than the conductive traces 406. In FIG. 4B, the surface roughness of the electrode contacts 407 has been increased to increase the electrically active stimulation surface 409. This may be done, for example, by depositing very small structures or particles on the stimulation surface 409 of the electrode contact 407.

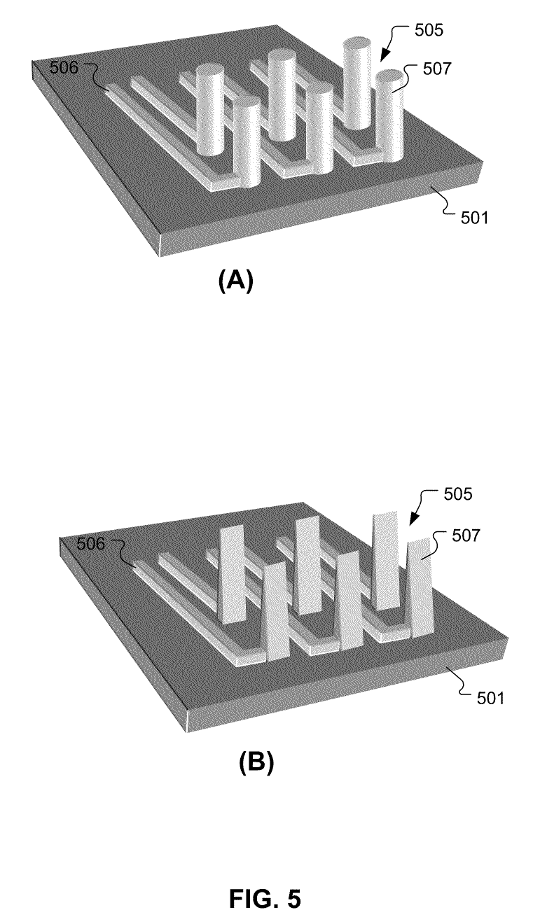

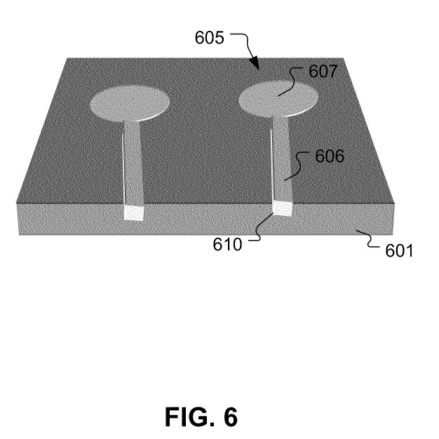

FIG. 5A-B shows various other three-dimensional stimulation electrodes 505 according to embodiments of the present invention. In FIG. 5A, the electrode contacts 507 are arranged in a two-dimensional array above the conductive trace elements 506 and the electrode substrate 501. In FIG. 5B, the electrode contacts 507 are arranged in a two-dimensional array and further are tapered (e.g. for penetration into the inserted tissue). In the specific case of penetrating electrode contacts 507, some or all of the electrode contacts 507 may be electro-polished or chemically etched to smooth their surface to minimize trauma during tissue penetration. Polishing may also be useful in other specific embodiments. FIG. 6 shows an embodiment of the present invention in which the stimulation electrodes 605 are embedded into recessed grooves 610 in the electrode substrate 601.

The processes described above can be useful for creating two-dimensional and/or three-dimensional structures for implantable stimulation electrodes. The resulting structures can be adjusted and optimized to have desired mechanical properties. And a finished electrode can be created by one continuous process without interruption to form in a single integrated structure. The design structure of the stimulation electrode can be changed simply by changing a CAD file where the geometrical parameters for the electrode design are defined (and possibly some basic process optimization).

Although various exemplary embodiments of the invention have been disclosed, it should be apparent to those skilled in the art that various changes and modifications can be made which will achieve some of the advantages of the invention without departing from the true scope of the invention. For example, in any of the foregoing, the stimulation electrode may specifically be a cochlear implant electrode. And embodiments of the present invention also include an implantable stimulation electrode formed by any of the foregoing methods.

What is claimed is:

1. A method of fabricating an implantable stimulation electrode comprising:
providing an arrangement of conductive metal powder atop an electrode substrate; and
focusing a laser beam on the metal powder to form the stimulation electrode in a given geometry with given dimensions and supported by the electrode substrate.

2. A method according to claim 1, wherein providing an arrangement of conductive metal powder includes blowing the metal powder onto the electrode substrate so that the stimulation electrode is formed by laser deposition of the metal powder.

3. A method according to claim 1, wherein providing an arrangement of conductive metal powder includes pre-layering the metal powder onto the electrode substrate so that the stimulation electrode is formed by laser sintering of the metal powder.

4. A method according to claim 1, wherein the given geometry is a two-dimensional geometry so that the stimulation electrode lies substantially in a single plane.

5. A method according to claim 1, wherein the given geometry is a three-dimensional geometry.

6. A method according to claim 1, wherein the stimulation electrode is formed in a recess in the surface of the electrode substrate.

7. A method according to claim 1, wherein the stimulation electrode is formed above the surface of the electrode substrate.

8. A method according to claim 1, wherein the stimulation electrode includes at least one electrode contact for delivering an electrical signal to adjacent tissue.

9. A method according to claim 8, wherein the electrode contact includes a layer of conductive surface structures for increasing the surface area of the electrode contact.

10. A method according to claim 8, wherein the stimulation electrode includes at least one conductive trace element for delivering the electrical signal to the electrode contact.

11. A method according to claim 10, wherein the stimulation electrode includes a plurality of conductive trace elements.

12. A method according to claim 10, wherein the electrode contact and the conductive trace element have the same height.

13. A method according to claim 10, wherein the electrode contact and the conductive trace element have different heights.

14. A method according to claim 1, further comprising:
smoothing at least one surface of the stimulation electrode to reduce insertion trauma.

15. A method according to claim 14, wherein the smoothing is based on electro-polishing.

16. A method according to claim 14, wherein the smoothing is based on chemical etching.

17. A method according to claim 1, wherein the stimulation electrode is formed into a plurality of different metal layers.

18. A method according to claim 1, wherein the metal powder includes at least one of iridium, gold, silver, palladium, tungsten, or an alloy thereof.

19. A method according to claim 1, wherein the metal powder includes platinum or a platinum alloy.

20. A method according to claim 1, wherein the electrode substrate is at least one of silicon, glass, polymer, or metal.

21. A method according to claim 1, wherein the stimulation electrode is a cochlear implant electrode.

22. An implantable stimulation electrode formed by a method according to any of claims 1-21.
